(19) United States
(12) Patent Application Publication
(10) Pub. No.: US 2004/0116824 A1
(43) Pub. Date: Jun. 17, 2004

(54) Evaluation or diagnostic kit

(75) Inventors: Jean-Pascal Hirt, Saint-Cloud (FR); Roland Jourdain, Meudon La Foret (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/674,491

(22) Filed: Oct. 1, 2003

Related U.S. Application Data
(60) Provisional application No. 60/428,933, filed on Nov. 26, 2002.

(30) Foreign Application Priority Data
Oct. 1, 2002 (FR) 02 12157

(51) Int. Cl. A61B 5/00; A61K 49/00

(52) U.S. Cl. 600/556; 424/9.2

(57) ABSTRACT

An evaluation or diagnostic kit includes a plurality of applicators containing test substances of various kinds. Each applicator can include a tube, a plug inside the tube, and at least one test substance inside the tube. The plug can be arranged, in use, to be expelled together with the substance when the substance leaves the inside space of the tube. A diagnostic or evaluation method is also provided.

### CROSS-REFERENCE TO RELATED APPLICATIONS

This document claims priority to French Application No. 02 12157, filed Oct. 1, 2002 and U.S. Provisional Application No. 60/428,933, filed Nov. 26, 2002, the entire content of both of which is hereby incorporated by reference.

### FIELD OF THE INVENTION

The present invention relates to diagnostic or evaluation kits, and particularly but not exclusively to those for use in evaluating the level of cutaneous neurosensitivity of an individual, or that individual's sensitivity to an allergen.

### BACKGROUND OF THE INVENTION

### Discussion of Background

U.S. Pat. No. 5,143,210 discloses a diagnostic kit including a support capable of receiving a plurality of flasks each containing a test chemical reagent. Each flask is relatively costly to make and the application of a test reagent requires the flask to be handled often. A flask can be accidentally knocked over while being handled. Too much reagent might possibly be taken out.

U.S. Pat. No. 3,958,571 discloses an applicator including a tube containing a liquid and having an applicator element at one end of the tube. Such an applicator is designed to apply medication such as a solution of iodine.

### SUMMARY OF THE INVENTION

There exists a need for a diagnostic or evaluation kit which is easy to transport, capable of being manufactured at relatively low cost, and simple to use.

In one embodiment, the invention provides a diagnostic or evaluation kit including a plurality of applicators containing test substances of various kinds and/or containing at least one compound at various different concentrations. Each applicator includes a tube, a plug inside the tube; and at least one test substance contained inside the tube. The plug can be arranged, in use, to be expelled together with the test substance when the test substance leaves the inside space of the tube. The term “test substance” is used to designate a substance serving to characterize, on its own or in combination with other substances or reagents, the state of an individual. According to the invention, various substances or various concentrations of a compound can be applied to the skin, including the mucous membranes, or to the hair or the finger- or toe-nails, for example, without the drawbacks connected with using flasks.

In addition, the quantity of test substance contained in each applicator may be easily kept down to a minimum needed for the desired application by appropriately selecting the dimensions of the tube. The volume of liquid contained in the tube can thus lie, for example, in the range from 0.01 milliliters (ml) to 5 ml, or preferably in the range from 0.05 ml to 1 ml.

An embodiment of the invention includes a diagnostic or evaluation kit made at a cost that is compatible with distribution on a large scale.

In an embodiment of the invention, the kit can include a plurality of applicators containing substances. The substances can include at least one compound at various different concentrations. For example, the kit can include at least two test substances containing at least one compound at concentrations varying by a factor lying in the range from 1.5 to 10, e.g. in the range from 2 to 5, from one applicator to another.

The test substances contained in the various applicators can contain a stimulating agent for stimulating the peripheral nervous system, for example.

The term “stimulating agent for stimulating the peripheral nervous system ” is used to designate a compound which makes it possible to induce a sensory response connected with activating sensory and cutaneous nerves having endings that flush with the stratum corneum epidermis.

The stimulating agent for stimulating the peripheral nervous system can be natural or synthetic capsaicinoids, preferably capsaicin, homocapsaicin, homodihydrocapsaicin, nordihydrocapsaicin, dihydrocapsaicin; lactic acid, glycolic acid, ethanol at a concentration greater than 50%, mustard seed oil, this list not being limiting. The stimulating agent can also include those disclosed in European patent EP 680,749, the entire content of which is hererby incorporated by reference.

In each test substance, the concentration of the stimulating agent for stimulating the peripheral nervous system may, for example, lie in the range from 10⁻⁶% to 10⁻²% by weight, relative to the total weight of the composition.

The kit can include at least one test substance that does not have any stimulating agent for stimulating the peripheral nervous system. Accordingly, a subject under test can compare the sensations felt when two test substances are applied, of which only one contains the stimulating agent for stimulating the peripheral nervous system.

In another embodiment of the invention, the kit can include a plurality of applicators containing test substances of various kinds. The test substances can, for example, contain respective allergens of various kinds.

The allergens can be, for example, allergens originating from acarids, animal hairs and scales, mold, hymenoptera venoms, foodstuffs, metals, in particular nickel, rubber, any compound that can be found in substances designed to be applied on the body or the hair, e.g. a compound used in hair coloring substances, in particular paraphenyldiamine (PPD), this list not being limiting.

The applicators can be packaged in various ways. The kit can, for example, include a housing including compartments in which the applicators are housed. The housing can, in particular, include at least one compartment configured to receive a single applicator, or in a variant, the housing can include at least one compartment configured to receive a plurality of applicators.

In yet another variant, the kit can include at least one bag for packaging at least one applicator, or a string of bags each containing at least one applicator.

Each applicator can include at least one mark representative of the kind of substance contained inside the tube, and/or of the concentration of a compound contained in the substance, e.g. at least one alphanumeric symbol and/or at least one color.

The inside space of the tube, in which the test substance is contained, may be defined at a second end, remote from the first, by a portion that can be broken off, removed, perforated, or deformed. When the break-off portion is broken off, the substance contained in the inside space of the tube can descend under gravity, expelling the plug of liquid or powder.

The applicator can be arranged in such a manner that after the break-off end has been broken off, the user can measure out the quantity of liquid that flows out by handling the tube as a pipette, while closing the top end of the tube with an index finger, and with the tube optionally sloping to a greater or lesser extent, where appropriate.

In an embodiment of the invention, the tube can be reclosed after only a fraction of the liquid contained inside it has moved out. Such reclosing can be performed, for example, using the break-off end. This end can be configured, for example, in such a manner as to be capable of constituting a closure plug. For example, the break-off end can include a spike suitable for engaging in the tube or on the tube in order to close it.

Where appropriate, the applicator can include a retaining element for retaining the break-off portion on the applicator after it has been broken off.

The tube can be provided at one end with an applicator element, the applicator element being separated from the test substance prior to the applicator being used, by the plug.

The applicator element can be porous, e.g. fibrous, e.g. in order to make it easy to impregnate with the test substance. For example, the applicator element can be a cotton bud, a foam bud, a felt tip, a flocked bud, and a tip made of ceramic or of sintered material, this list not being limiting.

In a variant, the tube does not have an applicator element. In this case, the tube can, for example, include an end that is configured so as to be able to scarify the skin.

As mentioned above, the plug can include a liquid and/or a powder. When the plug includes a liquid, the liquid can be, for example mineral oils, fluorine-containing substances, and silicones, this list not being limiting.

When the plug includes a powder, the powder can include organic or inorganic particles, which are solid or hollow, and can, for example be powders of microspheres of copolymers such as Expancel® (Nobel Industrie), of Nylon® (in particular Orgasol®), of waxes, of silicas, and of silicones, this list not being limiting.

The tube can be made of a transparent material, for example a transparent plastics material. With this embodiment, a user can observe the level of the substance inside the tube or observe its color, for example.

The tube can include a multilayer structure. At least one layer can form a barrier against air, e.g. a layer of varnish that is impermeable to air, or to a solvent, or to ultraviolet (UV) radiation.

Advantageously the test substance can be sterile. If desired, all the applicators of the kit can be completely sterilized.

In another embodiment, the invention also provides an evaluation method for evaluating the level of cutaneous neurosensitivity of an individual. The method can include a first step of applying, on a cutaneous zone of the individual, a first substance containing a physiologically acceptable vehicle and a stimulating agent for stimulating the peripheral nervous system. The substance can be applied using an applicator including a tube and a plug inside the tube. The substance can be contained in an inside space of the tube defined at a first end by the plug. The plug can be arranged, in use, to be expelled together with the substance when the substance leaves the inside space of the tube.

The method can further include a second step of gathering information representative of the detection, by the individual, of a dysesthetic sensation. If the individual does not detect any such sensation, the first and second steps can be repeated with a substance containing a higher concentration of the same agent until the individual detects a dysesthetic sensation, or until a substance at a maximum concentration (C4) of the agent is applied.

The method can include a step of deducing, from the last concentration applied, information regarding the cutaneous neurosensitivity of the individual.

The term “physiologically acceptable vehicle” is used to designate a vehicle that is compatible with the skin, the mucous membranes, the finger- or toe-nails, or the hair. It can be, for example, an aqueous solution, a water-alcohol solution, or an oil solution.

The cutaneous zone selected can be for example the bend of the arm, the earlobe, or the face, in particular the alae nasi, the furrow at the side of the nose, or the corner of the jawbone. A dysesthetic sensation can be the smallest painless sensation that is felt in the zone treated by the stimulating agent.

A cosmetic or care product can be prescribed as a function of the cutaneous neurosensitivity of the individual. The term “cosmetic” is used to designate a product as defined in EC Council Directive 93/35 of Jun. 14, 1993, amending EEC Directive 76/768 for the sixth time. The information deduced from the last concentration applied can be transmitted remotely, for example via the Internet.

In another embodiment, the invention also provides a diagnostic method for diagnosing an allergy. The method includes a step of depositing a substance containing an allergen on the skin or a mucous membrane of an individual. The substance can be deposited with an applicator including a tube, with a plug inside the tube. The substance can be inside the tube. The plug can be arranged, in use, to be expelled together with the substance when the substance leaves the inside of the tube.

The method can also include a step of deducing, from any possible cutaneous reaction of the individual, information concerning the sensitivity of the individual to the allergen under consideration.

In another embodiment, the invention also provides an evaluation method for evaluating a treatment substance. The method includes a step of performing the steps of one of the above-mentioned evaluation or diagnostic methods. The method further includes a step of applying the treatment substance. After a given number of applications and/or after a determined period of time has passed, the evaluation or diagnostic steps can be repeated. The effectiveness of the treatment carried out can be evaluated.

The invention also provides, independently or in combination with the above, a method of application of at least a substance contained in an inside space of the tube, the method including heating the tube with a heat source before application of the substance. The substance can include, for example, at least a thermoreversible thickener so that the substance is reduced into a fluid state when the heat increases. The heat source can be the human heat or a heat source outside the human body, for example a source of hot water.

Reducing the substance into a fluid state before application can improve the preservation of the substance during stocking in the tube, in particular by limiting the evaporation of the substance. Reducing the substance into a fluid state can also facilitate the passage of the substance through an application element, in particular an application element made of a porous material, for example cotton. The tube can, but need not, include a plug of one of a liquid and a powder disposed adjacent to the substance. The plug can be arranged to be expelled together with the substance when the substance leaves the inside space of the tube, in use.

### BRIEF DESCRIPTION OF THE DRAWINGS

Other characteristics and advantages of the invention will become apparent from the following detailed description, particularly when considered in conjunction with the drawings in which:

FIG. 1 is a diagram showing a diagnostic or evaluation kit in accordance with an embodiment of the invention;

FIGS. 2 to 4 show, in isolation, an applicator of the kit in FIG. 1;

FIGS. 5 and 6 show other examples of kits of the invention;

FIG. 7 shows an applicator packaged in an individual bag;

FIGS. 8 to 11 show various variant embodiments of the applicator element;

FIG. 12 shows an applicator that does not have an applicator element;

FIG. 13 is a fragmentary and diagrammatic view showing a variant of the applicator in FIG. 12;

FIGS. 14 and 15 show various embodiments of the break-off portion of the tube;

FIGS. 16 and 17 are diagrams showing applicators that include two-phase and multi-phase compositions respectively;

FIG. 18 is a diagrammatic and fragmentary view showing another example of an applicator;

FIG. 19 shows a receptacle that is able to receive an applicator; and

FIG. 20 shows another example of an applicator holder.

### DETAILED DESCRIPTION OF THE PREFERRED EMBODIMENTS

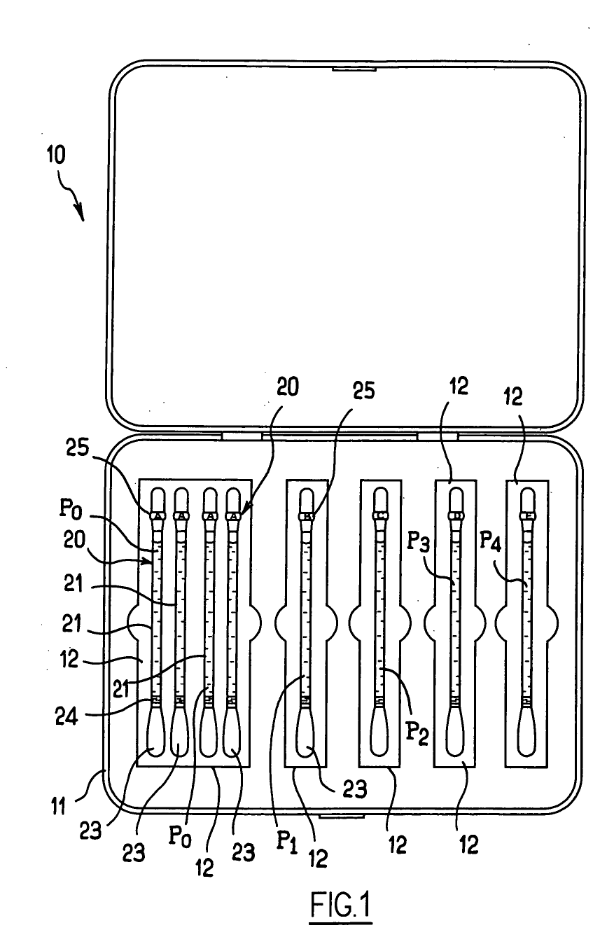

FIG. 1 shows an example of a diagnostic or evaluation kit 10 made in accordance with an embodiment of the invention. The kit 10 can include a box 11 with a plurality of compartments 12 each receiving an applicator 20 containing a test substance. By way of example, the test substances can enable the cutaneous neurosensitivity of an individual to be tested.

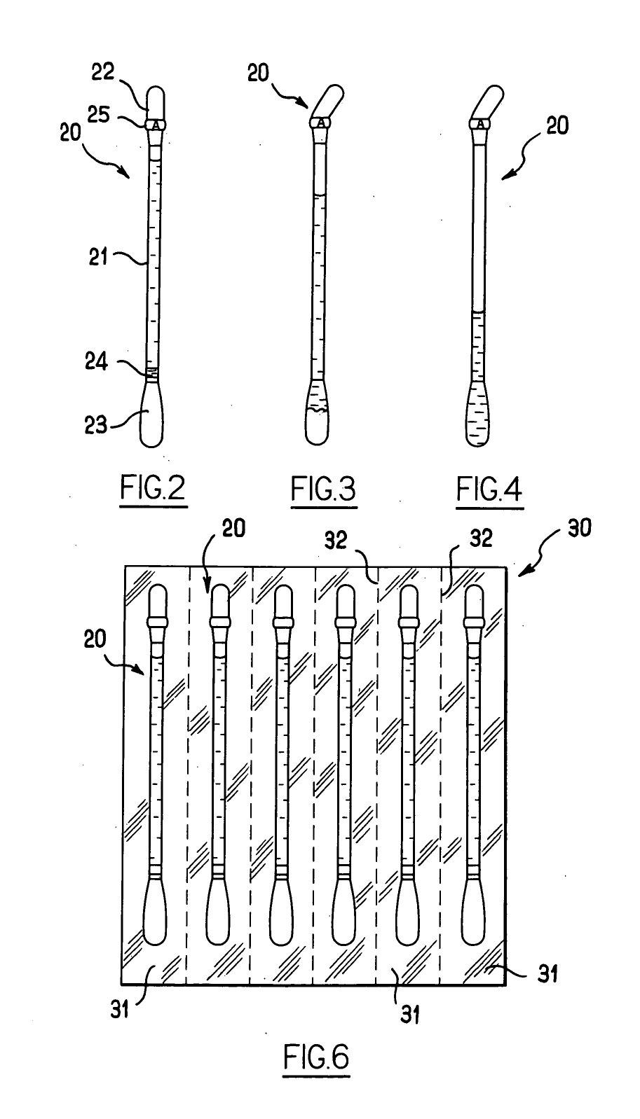

FIGS. 2 to 4 show an applicator 20 in isolation. By way of example, this applicator can be similar to the applicator as described in U.S. Pat. No. 5,702,035, the entire content of which is incorporated herein by reference. Applicators of that type are sold by the California supplier Swabplus Inc.

Each applicator 20 includes a tube 21 containing the test substance. The tube can be made, in the assembly shown, by extruding a transparent plastics material. The tube can be provided at a closed end with a break-off portion 22. In the example described, this portion is covered by a cotton bud. The tube 21 is open at its other end remote from the break-off portion 22, being provided at this end with an applicator element 23, for example. In the example shown, the applicator element 23 includes a cotton bud in the manner of a cotton applicator.

The test substance is contained in an inside space of the tube 21 situated between the break-off portion 22 and a plug 24 present in the tube 21 beside its open end. The volume of test substance can be suitable for a single use of the applicator. This volume can be determined as a function of the nature of the substance and of the applicator envisaged. It can lie in the range, for example, from 0.01 ml to 5 ml, and preferably in the range from 0.05 ml to 1 ml. The outside diameter of the tube 21 can be less than 6 millimeters (mm), for example, or even less than about 3 mm. The inside diameter of the tube 21 can lie in the range from about 0.5 mm to about 3 mm, for example.

The plug 24 can be any inert liquid or powder compatible with packaging the substance in the tube 21. For example, it can be a liquid which does not react with the test substance, is capable of being expelled easily from the tube 21 at the time of use, and which is also physiologically acceptable. The plug 24 can serve to isolate the test substance from air, preventing it from evaporating and preventing external contaminants from penetrating. The liquid of the liquid plug 24 can be, for example, a mineral oil or by a fluorine-containing substance, amongst other possibilities. In the example described, the liquid plug 24 is made of silicone. The quantity of liquid or powder forming the plug 24 is small compared with the quantity of test substance.

When the break-off portion 22 is broken off, air can penetrate into the tube 21 through its end remote from its open end. The substance can flow under gravity inside the tube 21, thereby reaching the applicator element 23, as shown in FIGS. 3 and 4, so as to be applied on the skin, for example. In the example described, the cotton bud covering the break-off portion 22 serves to hold it to the remainder of the tube 21, even after it has been broken off.

FIGS. 1 to 4 show that each applicator 20 includes a mark 25 that is visible to the user. The mark 25 can include a specific alphanumeric symbol or a color, for example, enabling the user to distinguish between different applicators.

The kit 10 can, for example, include a plurality of applicators 20 respectively containing different substances P₀, P₁, P₂, P₃, and P₄, each associated with a respective mark 25. In the example in FIG. 1, the housing contains eight applicators 20, but this is merely an example and the number of applicators can vary greatly without going beyond the scope of the present invention.

The substances P₀ to P₄ can be of different kinds or of the same kind. When the substances P₀ to P₄ are of the same kind, they can contain the same compound but have different respective concentrations.

The kit of the invention can also include a softening cream for treating the application zone, not shown in the figures, user instructions for the kit, and a note containing advice, where appropriate.

The kit 10 can, for example, be used as mentioned above, for evaluating the cutaneous sensitivity of an individual.

The various applicators 20 can thus contain for example capsaicin at various concentrations. For example, the substances P₀, P₁, P₂, P₃, and P₄ can contain capsaicin in a water-alcohol solution (10% ethanol, 90% water) having respective increasing concentrations by weight, e.g. equal to the following values: C₀=0% (vehicle only), C₁=3.3×10⁻⁵%, C₂=1×10⁻⁴%, C₃=3.3×10⁻⁴%, C₄=1×10⁻³%.

By way of example, a diagnosis is performed in the following way:

Step 1: Test substance of concentration C₀ is applied to the furrows on each side of the nose so as to accustom the subject to the sensations induced by applying the vehicle. A wait of three minutes then follows.

Step 2: The vehicle only is applied to one side, and capsaicin solution at a concentration C₁ is applied to the other side. After three minutes the subject is asked if any difference can be felt between the two sides. If the subject describes a dysesthetic sensation on the side that has received the capsaicin, then the test is stopped and C₁ is taken to be the detection threshold. If not, the following step is performed.

Step 3: Solution C₂ is applied to the side that received the vehicle in step 1, and the vehicle only is applied to the other side. After three minutes the subject is asked if any difference can be felt between the two sides. If the subject describes a particular sensation on the side that has received the capsaicin, then the test is stopped and C₂ is taken to be the detection threshold. If not, step 4 is performed.

Step 4: Solution C₃ is applied to the side that received the vehicle in step 2, and the vehicle only is applied to the other side. After three minutes the subject is asked if any difference can be felt between the two sides. If the subject describes a particular sensation on the side that has received the capsaicin, then the test is stopped and C₃ is taken to be the detection threshold. If not, step 5 is performed.

Step 5: Solution C₄ is applied to the side that received the vehicle only in step 3, and the vehicle only is applied to the other side. After three minutes the subject is asked if any difference can be felt between the two sides. If the subject describes a particular sensation on the side that has received the capsaicin, C₄ is taken to be the detection threshold. If not, a concentration greater than C₄ is taken to be the detection threshold.

If the individual does not detect any dysesthetic sensation after applying all the applicators 20, the individual is declared as having skin that is not sensitive or that is poorly sensitive. If a dysesthetic sensation is detected after applying the substance P₁, P₂, or P₃, the individual is declared as having excessively sensitive skin, very sensitive skin, or fairly sensitive skin, respectively.

It can be considered that the individual has very high neurosensitivity if the capsaicin concentration of the substance detected by the individual lies in the range from 1×10⁻⁶% to 1×10⁻⁴%, fairly high if it lies in the range from 1.5×10⁻⁴% to 0.75×10⁻³%, low if it lies in the range from 1.10×10⁻³% to 5.10×10⁻³%, and very low if it is about 1×10⁻²%.

It should be observed that between two successive substances P₀, P₁, P₂, P₃, and P₄, the concentration increases by a factor of about √10, but it can increase by some other factor without going beyond the scope of the present invention, e.g. by a factor lying in the range 1.5 to 10, and preferably in the range 2 to 5.

Naturally, the applicators 20 can be packaged in another way. FIG. 5 shows a variant of the FIG. 1 kit, in which the housing 11 includes a plurality of compartments 17 each capable of receiving a plurality of applicators 20 all containing an identical test substance.

The housing can include a marker 18 associated with each compartment 17 so as to enable the user to identify the kind of substance contained in the applicators present in the corresponding compartment 17.

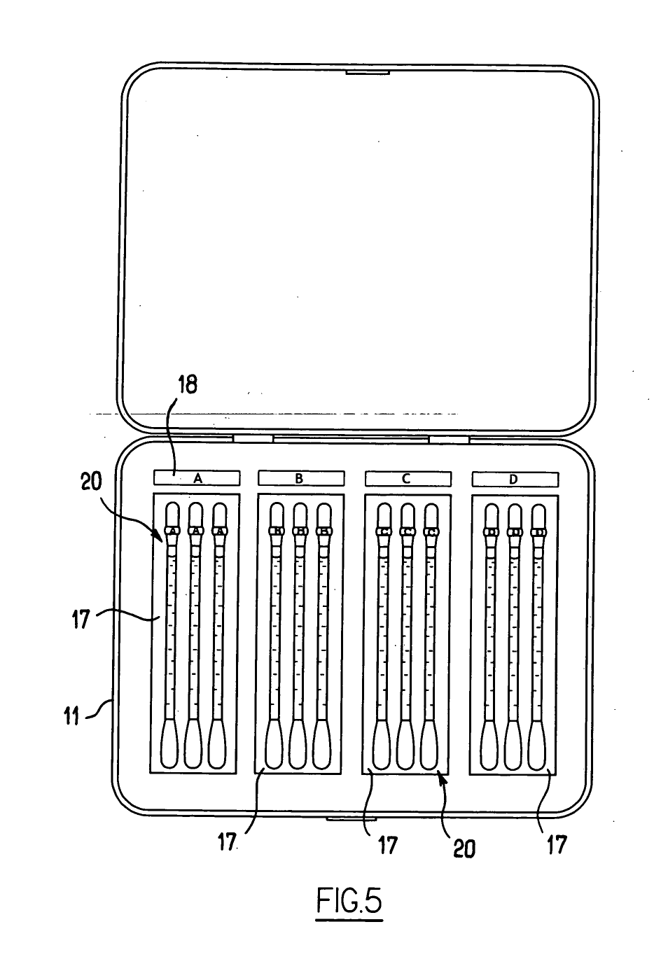

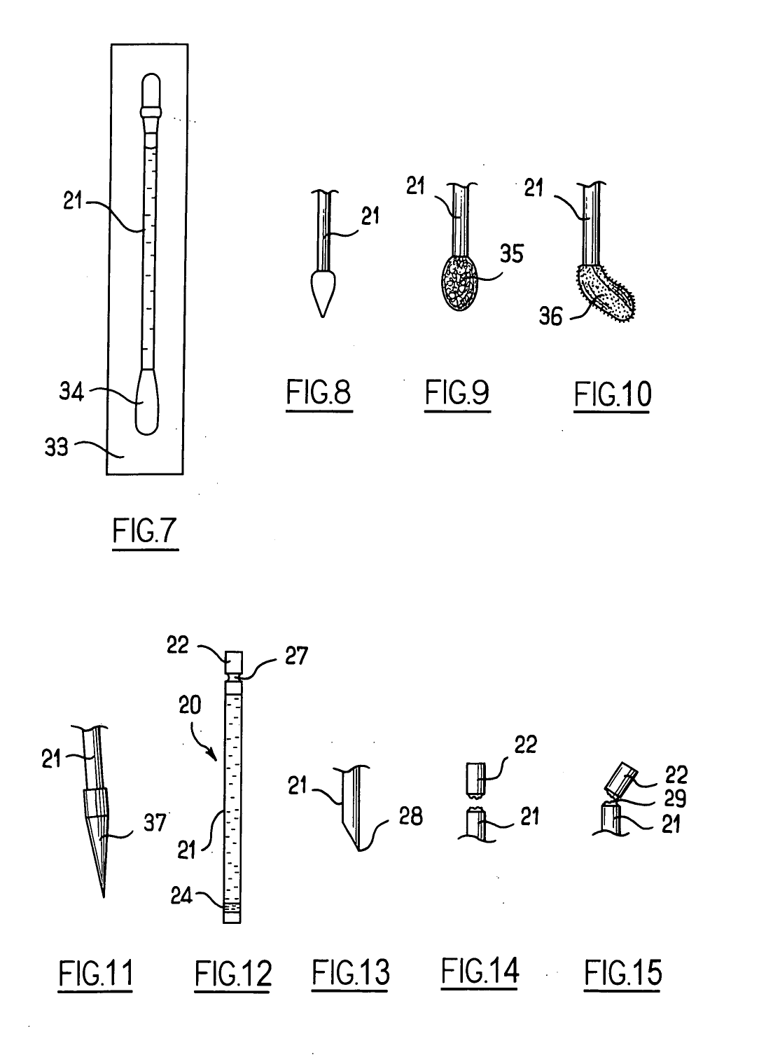

The applicators 20 can be packaged other than in a housing. By way of example, FIG. 6 shows a kit comprising a string 30 of bags 31 each containing one applicator 20, for example. Each applicator can be sterile. The applicators 20 can also be packaged in individual bags 33, as shown in FIG. 7.

Where appropriate, each applicator 20 can include an applicator element that has been pre-impregnated with a particular compound 34 or with a liquid prior to use. When the applicator element has been pre-impregnated with a liquid compound 34, the applicator 20 is advantageously made available in sealed packaging such as the bag shown in FIG. 7, for example.

By flowing into the applicator element, the substance contained in the tube 21 can dissolve the compound 34 which impregnates the applicator element 23, or it can react with the compound. This makes it possible to have an applicator including at least two compounds that cannot be packaged together and that must be mixed together extemporaneously.

An applicator 20 can include an applicator element of a shape other than that of a conventional cotton bud. By way of example, the applicator element can be of tapering shape, as shown in FIG. 8.

By way of example, the applicator element can also be made of any porous material, e.g. a fibrous material, and it can optionally be elastically compressible. By way of example, and as shown in FIG. 9, the applicator element can be in the form of a foam bud 35.

The applicator element can include flocking 36 on its surface, as shown in FIG. 10. This figure also shows that the applicator element can be curved in shape, with a portion extending along a longitudinal axis that does not coincide with the axis of the tube 21.

By way of example, the applicator can also be in the form of a tip 37 that is of tapering shape, in particular a felt tip, as shown in FIG. 11. The bud can be made of a porous material, or, in a variant, of a material that is not porous but that includes at least one internal channel or groove that enables the test substance contained in the tube 21 to flow towards the distal end.

The applicator 20 can also be free of an applicator element, i.e., the applicator 20 does not have an applicator element, as shown in FIG. 12. Such an embodiment can be useful, in particular when the substance contained in the tube 21 must be deposited in the form of a drop on the surface of the skin, for example. The tube 21 can thus be made with a tapering shape at the end remote from the break-off portion 22, e.g. with a chamfered shape, as shown in FIG. 13. A point 28 can thus be formed at the end of the tube 21, enabling the skin to be lightly scarified. Such scarification can be useful if it is desired to evaluate the sensitivity of an individual to various allergens by performing a skin test.

Using this embodiment, the skin can be lightly scarified by the point 28. The break-off portion 22 can then be broken off so as to enable the test substance contained in the tube 21 to flow over the zone of the skin including the scarification.

A cutaneous reaction observed after several minutes or several hours, or even several days, can give information on the sensitivity of the individual to the allergen under consideration.

In another embodiment, if the shape of the end of the applicator allows it, it is also possible to use the applicator to perform a prick-test. The test substance contained in the tube 21 can be deposited on the skin, and then the skin can be pricked with the point 28 through the substance deposited on its surface, so as to make it easier to penetrate into the dermis.

The test substance contained in the applicators 20 can contain various allergens. It can, in particular, make it possible to know the sensitivity of an individual to allergens such as those originating from acarids, animal hairs and scales, mold, hymenoptera venoms, foodstuffs, metals, rubber, this list not being limiting.

FIG. 12 shows that the break-off portion can be connected to the remainder of the tube via a preferred breakage zone 27, e.g. implemented in the form of a thinning in the wall of the tube 21 at this level. The break-off portion can be made in various other manners without going beyond the scope of the present invention.

In particular, as shown in FIG. 14, the applicator can be configured in such a manner that the break-off portion 22 is capable of being completely separated from the tube 21 after manually applying a breaking movement by holding the tube 21 in one hand and the break-off portion 22 between two fingers of the other hand.

The applicator can alternatively be configured in such a manner that, after use, the break-off portion 22 remains connected to the tube 21 via a bridge of material 29, as shown in FIG. 15. When the break-off end is capable of being completely detached from the tube, the applicator can be used as a pipette, for example, with the user being capable of closing the top end with a finger in order to dispense the liquid into the applicator element in a controlled manner, e.g. drop by drop.

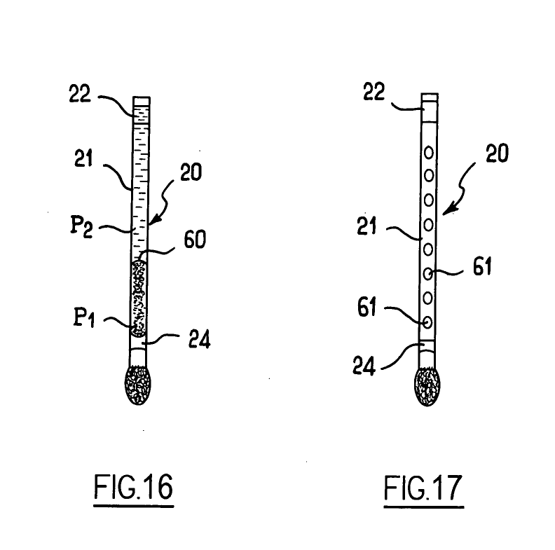

The tube of the applicator can include one or more test substances. FIG. 16 shows a tube including two liquid test substances P₁ and P₂ present in the form of two phases each occupying a fraction of the length of the tube. The two substances P₁ and P₂ are in contact with each other via an interface 60.

One of the substances can also be present in the form of at least one globule within the other phase, for example in the form of a plurality of globules 61 as shown in FIG. 17. This can make it possible, for example, to measure out the liquid using the tube as a pipette, or to improve the appearance of the applicator.

A plurality of different liquids can also be dispersed in the form of a plurality of globules in a single phase. The various globules can thus correspond to test substances containing reagents at different concentrations and/or of different kinds.

When contained in the tube, it is also possible for the substance P₂ to be solid, e.g. being constituted by a powder that is soluble in the liquid P₁. The substances P₁ and P₂ can be separated from each other prior to use by a plug. The volume of the substance P₂ can be small enough to ensure that the substance P₂ can be dissolved easily in use.

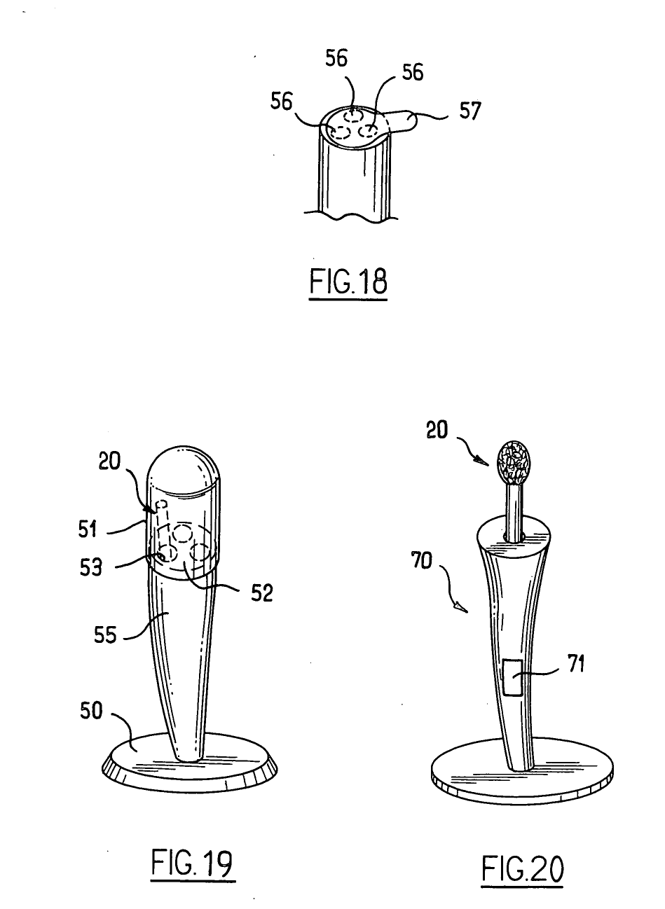

In the examples described above, the tube includes a single internal channel, but it would not go beyond the scope of the present invention for the tube to include multiple channels. FIG. 18 shows the top end of a tube that contains three internal channels 56 by way of example, each containing a respective liquid and an associated plug, the tube being closeable at this end prior to use by a removable capsule 57 which can be stuck or heat-sealed on the tube, for example.

FIG. 19 shows a receptacle suitable for receiving an applicator prior to or after use. By way of example, such a receptacle can include a stand 50 supporting a body 55 whose top end is configured to enable a closure cap 51 to be fixed in place to close the receptacle in substantially leaktight manner. A support element 52 is disposed inside the body 55 and has at least one orifice 53 enabling an applicator 20 to be engaged therein.

Thus, if so desired, after use, the user can place the applicator in the receptacle so that it can be reused subsequently. The presence of the cap 51 makes it possible to avoid the applicator element drying out, for example.

It is also possible to use, in association with at least one applicator, a support 70 of the kind shown in FIG. 20, enabling the applicator to be held with its applicator element visible. This support can include, for example, means 71 enabling the break-off end to be broken off when the applicator is put into place in the support 70. By way of example, these means 71 include a window giving access to the break-off end, or an element that is movable relative to the support and which applies lateral thrust on the break-off end when actuated. The tube need not have a break-off end but can merely have an end that is closed, with the support 70 being fitted with a blade or a spike, for example, serving to cut or pierce the tube so as to allow air to penetrate into the inside, and the liquid and the plug to be expelled when the applicator is used.

Naturally, the invention is not limited to the embodiments described above. The end of the tube 21 remote from the end through which the test substance leaves can, for example, be closed other than by a portion suitable for being broken off manually. For example, it can be closed by a plug or a piston in one of the ways represented in FIGS. 3 to 8 of U.S. Pat. No. 3,958,571, the content of which is incorporated herein by reference. The tube 21 can be made with an enlarged portion, e.g. in order to enable pressure to be exerted on the liquid so as to cause it to leave the tube.

The term “tube” is used to cover any body that is preferably generally elongate and having a section with at least one internal channel capable of containing a liquid. A tube can optimally have a constant section. A tube can present a longitudinal axis that is rectilinear or otherwise. The invention is not limited to a tube of circular outside section, nor is it limited to a tube made in accordance with the teaching of U.S. Pat. No. 5,702,035.

It is possible to test the cutaneous neurosensitivity of an individual, as described above, or indeed to test any type of biological, chemical, thermal, or mechanical sensitivity of the skin, of a mucous membrane, of the hair or of the finger- or toe-nails.

Throughout the description, including in the claims, terms such as comprising, including, having, or has should be understood as being synonymous with “comprising at least one” or “including at least one,” unless specified to the contrary.

Obviously, numerous modifications and variations of the present invention are possible in light of the above teachings. It is therefore to be understood that, within the scope of the appended claims, the invention may be practiced otherwise than as specifically described herein.

What is claimed as new and desired to be secured by Letters Patent of the United States is:

1. An evaluation or diagnostic kit comprising a plurality of applicators containing different test substances, each applicator comprising:
a tube;
a plug inside the tube; and
at least one test substance contained in an inside space of the tube defined at a first end by the plug, the plug being arranged, in use, to be expelled together with the test substance when said test substance leaves the inside space of the tube.

2. The kit according to claim 1, wherein the test substances comprise different allergens.

3. The kit according to claim 2, wherein the allergens are selected from the group consisting of allergens originating from acarids, animal hairs and scales, mold, hymenoptera venoms, foodstuffs, metals, rubber, any compound that can be found in substances designed to be applied on the body or the hair.

4. The kit according to claim 1, further comprising a housing including compartments in which the applicators are housed.

5. The kit according to claim 4, wherein the housing includes at least one compartment configured to receive a single applicator.

6. The kit according to claim 4, wherein the housing includes at least one compartment configured to receive a plurality of applicators.

7. The kit according to claim 1, further comprising at least one bag for packaging at least one applicator.

8. The kit according to claim 7, further comprising a string of bags each containing at least one applicator.

9. The kit according to claim 1, wherein each applicator includes at least one mark corresponding to at least one of a type of test substance inside the tube and a concentration of a compound contained in the test substance.

10. The kit according to claim 9, wherein the mark comprises at least one of an alphanumeric symbol and a color.

11. The kit according to claim 1, wherein the test substance in the tube has a volume in a range from 0.01 ml to 5 ml.

12. The kit according to claim 1, wherein test substance in the tube has a volume in a range from 0.05 ml to 1 ml.

13. The kit according to claim 1, wherein the inside space is defined at a second end, remote from the first, by a breakable portion.

14. The kit according to claim 1, wherein the inside space is defined at a second end, remote from the first, by a removable portion.

15. The kit according to claim 1, wherein the inside space is defined at a second end, remote from the first, by a perforatable portion.

16. The kit according to claim 1, wherein the inside space is defined at a second end, remote from the first, by a deformable portion.

17. The kit according to claim 13, wherein the applicator includes a retaining element for retaining the breakable portion on the applicator after it has been broken off.

18. The kit according to claim 1, wherein the tube is provided at one end with an applicator element, the applicator element being separated from the test substance prior to use, by the plug.

19. The kit according to claim 18, wherein the applicator element is selected from the group consisting of a cotton bud, a foam bud, a felt tip, a flocked bud, and a tip made of ceramic or of sintered material.

20. The kit according to claim 1, wherein the tube is free of an applicator element.

21. The kit according to claim 20, wherein the tube includes an end configured so as to be able to scarify the skin.

22. The kit according to claim 1, wherein the plug comprises a liquid, and wherein said liquid is selected from the group consisting of mineral oils, fluorine-containing substances, and silicones.

23. The kit according to claim 1, wherein the plug comprises a powder, and wherein said powder is selected from the group consisting of powders of microspheres of copolymers, of Nylon®, of waxes, of silicas, and of silicones.

24. The kit according to claim 1, wherein the plug comprises one of a liquid and a powder.

25. The kit according to claim 3, wherein said allergen comprises nickel.

26. A diagnostic method for diagnosing an allergy, the method comprising the steps of:
deposing a substance containing an allergen on the skin or a mucous membrane of an individual, the substance being deposited with an applicator comprising a tube, a plug inside the tube, the substance being contained in an inside space of the tube defined at a first end by the plug, the plug being arranged, in use, to be expelled together with the substance when said substance leaves the inside space of the tube; and
deducing, from any possible cutaneous reaction of the individual, information concerning the sensitivity of the individual to the allergen under consideration.

27. An evaluation method for evaluating a treatment substance, the method comprising the steps of:
performing the steps of the method according to claim 26;
applying the treatment substance;
repeating the steps of the method according to claim 26; and
evaluating an effectiveness of the treatment substance.

28. The method of claim 27, wherein the repeating step is performed after a number of applications.

29. The method of claim 27, wherein the repeating step is performed after a determined period of time has passed,

30. An evaluation or diagnostic kit comprising a plurality of applicators containing test substances with at least one compound at different concentrations, each applicator comprising:
a tube;
a plug inside the tube; and
at least one test substance in an inside space of the tube defined at a first end by the plug, the plug being arranged, in use, to be expelled together with the test substance when said test substance leaves the inside space of the tube.

31. The kit according to claim 30, comprising at least two test substances with at least one compound at concentrations varying by a factor of at least two from one applicator to another.

32. The kit according to claim 30, wherein at least one substance comprises a stimulating agent for stimulating a peripheral nervous system.

33. The kit according to claim 32, wherein the stimulating agent for stimulating the peripheral nervous system is selected from the group consisting of natural or synthetic capsaicinoids, homocapsaicin, homodihydrocapsaicin, nordihydrocapsaicin, dihydrocapsaicin; lactic acid, glycolic acid, ethanol at a concentration greater than 50%, mustard seed oil.

34. The kit according to claim 33, wherein said stimulating agent comprises capsaicin.

35. The kit according to claim 32, wherein a concentration of the stimulating agent for stimulating the peripheral nervous system lies in a range from 10-6% to 10-2% by weight.

36. The kit according to claim 30, further comprising a housing including compartments in which the applicators are housed.

37. The kit according to claim 36, wherein the housing includes at least one compartment configured to receive a single applicator.

38. The kit according to claim 36, wherein the housing includes at least one compartment configured to receive a plurality of applicators.

39. The kit according to claim 30, further comprising at least one bag for packaging at least one applicator.

40. The kit according to claim 39, further comprising a string of bags each containing at least one applicator.

41. The kit according to claim 30, wherein each applicator includes at least one mark corresponding to at least one type of test substance inside the tube and a concentration of the compound in the test substance.

42. The kit according to claim 41, wherein the mark comprises at least one of an alphanumeric symbol and a color.

43. The kit according to claim 30, wherein the test substance in the tube has a volume in a range from 0.01 ml to 5 ml.

44. The kit according to claim 30, wherein the test substance in the tube has a volume in a range from 0.05 ml to 1 ml.

45. The kit according to claim 30, wherein the inside space is defined at a second end, remote from the first, by a breakable portion.

46. The kit according to claim 30, wherein the inside space is defined at a second end, remote from the first, by a removable portion.

47. The kit according to claim 30, wherein the inside space is defined at a second end, remote from the first, by a perforatable portion.

48. The kit according to claim 30, wherein the inside space is defined at a second end, remote from the first, by a deformable portion.

49. The kit according to claim 45, wherein each applicator includes a retaining element for retaining the breakable portion on the applicator after it has been broken off.

50. The kit according to claim 30, wherein the tube is provided at one end with an applicator element, the applicator element being separated from the test substance prior to use, by the plug.

51. The kit according to claim 50, wherein the applicator element is selected from the group consisting of a cotton bud, a foam bud, a felt tip, a flocked bud, and a tip made of ceramic or of sintered material.

52. The kit according to claim 30, wherein the tube is free of an applicator element.

53. The kit according to claim 52, wherein the tube includes an end configured so as to be able to scarify the skin.

54. The kit according to claim 30, wherein the plug comprises a liquid, and wherein said liquid is selected from the group consisting of mineral oils, fluorine-containing substances, and silicones.

55. The kit according to claim 30, wherein the plug comprises a powder, and wherein said powder is selected from the group consisting of powders of microspheres of copolymers, of Nylon® of waxes, of silicas, and of silicones.

56. The kit according to claim 30, wherein said plug comprises one of a liquid and a powder.

57. An evaluation method for evaluating a level of cutaneous neurosensitivity of an individual, the method comprising the steps of:
1) applying, on a cutaneous zone of the individual, a substance containing a physiologically acceptable vehicle and a stimulating agent for stimulating the peripheral nervous system, the substance being applied using an applicator comprising a tube, a plug inside the tube, the substance being contained in an inside space of the tube defined at a first end by the plug, the plug being arranged, in use, to be expelled together with the substance when said substance leaves the inside space of the tube;
2) gathering information representative of a detection, by the individual, of a dysesthetic sensation;
3) if the individual does not detect any such sensation, repeating steps 1) and 2) with a substance containing a higher concentration of a same agent until the individual detects a dysesthetic sensation, or until a substance at a maximum concentration of said agent is applied; and
4) deducing, from a last concentration applied, information regarding the level of cutaneous neurosensitivity of the individual.

58. An evaluation method for evaluating a treatment substance, the method comprising the steps of:
performing the steps of the method according to claim 57;
applying the treatment substance;
repeating the steps of the method according to claim 57; and
evaluating an effectiveness of the treatment substance.

59. The method of claim 58, wherein the repeating step is performed after a number of applications

60. The method of claim 58, wherein the repeating step is performed after a determined period of time has passed,

61. A system for evaluating a level of cutaneous neurosensitivity, comprising:
a packaging; and
a plurality of containers provided in said packaging, each container comprising:
a first end which is open,
a second end which is closed in a first position,
a removable plug provided inside said container in said first position, said removable plug defining a closed volume between said plug and said second end in said first position, and
a stimulating agent in said volume in said first position,
wherein said second end is movable to a second position which is open, said stimulating agent being in communication with an outside of said container via said first open end in said second position; and
wherein at least two of said containers have different concentrations of said stimulating agent.

62. The system of claim 61, wherein a first container comprises a first stimulating agent and a second container comprises a second stimulating agent different from said first stimulating agent.

63. The system of claim 61, wherein said plug is outside of said container in said second position.

64. The system of claim 61, wherein said plug is expelled with said allergen in said second position.

65. The system of claim 61, wherein said containers are tubes.

66. The system of claim 61, wherein each container is coupled to an applicator, said applicator covering said first end.

67. The system of claim 66, wherein said applicator comprises a porous material.

68. The system of claim 61, wherein said first end has a chamfered shape.

69. The system of claim 61, wherein said packaging is a box.

70. The system of claim 61, wherein said packaging comprises a plurality of bags, each of said containers being provided in one of said bags.

71. A system for evaluating a sensitivity, comprising:
a packaging; and
a plurality of tubes provided in said packaging, each tube comprising:
a first end which is open,
a second end which is closed in a first position,
a substance in said volume in said first position,
wherein said second end is movable to a second position which is open, said substance being in communication with an outside of said container via said first open end in said second position, and
wherein said second end is attached to said tube in, said second position.

72. The system of claim 71, wherein said substance is an allergen.

73. The system of claim 71, wherein said substance is a stimulating agent.

74. The system of claim 71, wherein each of said tube further comprises a removable plug provided inside said tube in said first position, said removable plug defining a closed volume between said plug and said second end in said first position.

75. The system of claim 71, wherein each of said tube further comprises a thermoreversible thickener inside said volume.

76. The system of claim 71, wherein said packaging comprises a box.

77. The system of claim 71, wherein said packaging comprises a plurality of bags, each of said tubes being in one of said bags.

78. The system of claim 71, wherein said packaging comprises:
a stand; and
a body mounted on said stand.

79. The system of claim 78, wherein each of said tubes has a portion extending outside said body, and wherein said packaging further comprises a closure cap coupled to said body and over said portions.
